(19) United States
(12) Patent Application Publication
(10) Pub. No.: US 2015/0025600 A1
(43) Pub. Date: Jan. 22, 2015

(54) LASER-BASED TREATMENT FOR MALARIA

(71) Applicant: Eitan Zvi Gross, Rogers, AR (US)

(72) Inventor: Eitan Zvi Gross, Rogers, AR (US)

(21) Appl. No.: 14/331,212

(22) Filed: Jul. 14, 2014

Related U.S. Application Data
(60) Provisional application No. 61/856,281, filed on Jul. 19, 2013.

(51) Int. Cl. A61N 5/06; A61M 1/36; A61M 1/14

(52) U.S. Cl. CPC A61N 5/062; A61M 1/14; A61M 1/3681; A61N 2005/0659; A61N 2005/067
USPC 607/89; 210/251; 210/646

(57) ABSTRACT

Malaria, caused by the parasite Plasmodium, is a devastating disease killing more than 800,000 people a year worldwide. Plasmodium replicates within erythrocytes by digesting hemoglobin, producing haemozoin as a byproduct which accumulates within the parasite. The development of vaccines is hampered by lack of memory immune response, while the long-term effectiveness of current anti-malaria drugs is limited due to the emergence of drug-resistant strains. Furthermore, people who are deficient of the enzyme glucose 6-phosphate dehydrogenase exhibit fatally-adverse drug effects. To overcome these hurdles, I propose a novel laserbased, non-pharmacological treatment for malaria. The treatment is based on the ability of haemozoin to convert light in the near infra-red into ultra-violet (UV) radiation via Third Harmonic Generation. The UV light produced by haemozoin can in turn kill the parasite. In experiments with infected erythrocytes we obtained a 4-log reduction in parasetemia following six passes of the blood through the laser beam.

### CROSS-REFERENCE TO RELATED APPLICATION

This application claims the benefit of U.S. Provisional Patent Application No. 61/856,281 filed Jul. 19, 2013 which is incorporated herein in its entirety.

### BACKGROUND

Malaria is a devastating disease killing more than 800,000 people a year worldwide(1). Malaria is caused by the parasite Plasmodium vectored by mosquitoes. The parasite infects erythrocytes where it replicates(2). The development of human vaccines is hampered by a complex intra-erythrocytic eukaryote pathogen and lack of a persistent memory immune response to malaria. Due to the chronic nature of some Plasmodium strains, both T cells and B cells become less functional. Furthermore, Plasmodium has several life stages, making selection of important antigens for targeting in a vaccine more challenging (3). Several classes of drugs are currently in use to treat malaria. These include quinolines, antifolates, and a rtemisinin-combination therapy (ACTs). Quinolines are haemozoin inhibitors which bind to purified haem and associate with haemozoin-containing fractions from Plasmodium, inhibiting the conversion of haem to haemozoin(4). Antifolates block folic acid synthesis, which is essential to Plasmodium growth because the parasite is unable to utilize pyrimidines already synthesized by the host and must use this pathway to make its own. Artemisinins, are activated by haem or free iron to generate parasiticidal radicals. Unfortunately, the long-term efficacy of the quinolines and antifolates has been limited due to the fast emergence of drug-resistant Plasmodium strains(S). Furthermore, malaria has been implicated in the spreading of glucose 6-phosphate dehydrogenase (G6PD)-deficient carriers in malaria endemic countries(6, 7). This X-chromosome linked mutation confers resistance to the disease upon its carrier, but at the same time also renders them fatally-intolerant to current anti-malaria drugs(8).

### BRIEF SUMMARY OF THE INVENTION

Recent studies have now shown that Plasmodium falciparum (the most deadly strain of the parasite that causes malaria) can adapt itself to grow in G6PD-deficent red blood cells(9), thus denying the carriers of this mutation the natural protection against the disease, leaving them as the most vulnerable population to the perils of the parasite. To overcome this hurdle and the other obstacles faced by pharmacological interventions, as outlined in the Background section, I propose here a laser-based, non-pharmacological treatment for malaria. The new treatment modality is based on the ability of haemozoin, naturally present within the parasite, to convert light in the near infra-red (NIR) region of the electromagnetic spectrum into ultra-violet (UV) radiation via a non-linear optical process known as Third Harmonic Generation (THG). Hence, by irradiating the parasite in the blood of an infected person with the laser, haemozoin can be turned into a “localized” source of UV radiation that can kill the parasite. UV light has been shown to offer an effective germicidal treatment against a broad range of pathogens including viruses(10), bacteria(11), fungi(12) and protozoa(13). Haemozoin exhibits a very strong THG signal(14, 15). In THG, a compound converts three photons of the laser light within the focus of a laser beam into one emitted photon of triple the frequency. The proposed treatment requires illuminating the parasite with a laser, either directly through the skin or by attaching the patient to a dialysis machine and passing the blood through narrow tubing equipped with a NIR-transparent window through which the blood can be irradiated.

### BRIEF DESCRIPTION OF THE DRAWINGS

The foregoing and other objects, features and advantages will be apparent from the following description of particular embodiments of the present disclosure, as illustrated in the accompanying drawings in which like reference characters refer to the same parts throughout the different views. The drawings are not necessarily to scale, emphasis instead being placed upon illustrating the principles of various embodiments of the present disclosure.

FIG. 1 shows the principle of third harmonic generation (THG). (top panel) A black-box diagram illustrating the principle of THG. ωₒ is the fundamental laser frequency. (bottom panel) Optical transitions between the different energy levels are indicated by gray arrows. The darker the shade of the arrow the larger the probability of that transition. The frequency of light emitted by THG is 3ω₀. NLOC, non-linear optical crystal (e.g. haemozoin).

FIG. 2 shows the dependence of the logarithm of light intensity emitted by 10 microM haemozoin in solution (in units of fW/cm²) as a function of the logarithm of incident laser light intensity (in units of mW/cm²). The data were fitted by a straight line with a slope of 2.81±0.3.

FIG. 3 shows growth curves for late-phase trophozoite Plasmodium falciparum in a blood sample that was passed through a NIR laser beam for the indicated number of times and then propagated using the continuous culture method, as described below. Ordinate represents the logarithm of the number of parasitized red bold cells, pRBC (N) divided by the initial number of pRBC (Nₒ).

FIG. 4 illustrates dose-response curves for NIR laser-induced inactivation of Plasmodium falciparum. The logarithm of ratio of number of un-irradiated parasites at the indicated phase to the number of parasites left after exposure to the laser; plotted against the number of times the entire blood sample passed through the laser beam, on a log-linear graph. Each data point represents the mean of three repetitions and the bars represent SEM. Data points with asterisks represent the number of pass-through times for which the mean value of log (Nₒ/N) for ring phase parasite was statistically different (p<0.05) from the corresponding value obtained for early and late phase parasites. ES, early trophozoites; S, late trophozoites.

FIG. 5A presents laser-induced bactericidal effect of haemozoin. Escherichia coli colonies on agar plates following 0 (upper row), 20 (middle row) and 40 (lower row) minutes of irradiation in the presence of 1 microM synthetic haemozoin.

FIG. 5B presents a dose-response curves for NIR laser-induced inactivation of E. coli in the presence of synthetic haemozoin. The ratio of number of bacteria left after exposure to laser to the number of un-irradiated bacteria; plotted against irradiation time on a log-linear graph. Also shown is NIR laser-induced killing of E. coli in the presence of 10 microM hemin. Each data point represents the mean of four repetitions and the bars represent SEM. Data points with asterisks represent exposure times for which the mean value of log (Nₒ/N) in the presence of haemozoin was statistically different (p<0.05) from the corresponding value obtained in the presence of hemin.

Table 1: Dose-response data for NIR laser germicidal effects.

### DETAILED DESCRIPTION

Light propagating through a vacuum will obey the principle of superposition, however this is not generally true for light propagating through condensed media. As light propagates through transparent media, it induces a dipole moment on any atoms present in the propagating electromagnetic field. At sufficiently high field strengths, the induced dipole moment is no longer proportional to the applied field, thus giving rise to nonlinear optical effects.

The dipole per unit volume (called the polarization), can generally be expressed by Eqn

P=ε₀V(r)E (1)

where ε₀ is the permittivity of free-space, V(r) is the restoring force acting on the polarized medium as a function of electron displacement from the nucleus and E is the electric field within the light wave. If V(r) is perfectly linear, then;

P=ε₀(1+ε)E (2)

where ε is the permittivity of the medium, and is related to the refractive index (n) at optical frequencies by;

ε=n² (3)

At low field strengths, a linear approximation of V(r) is suitable and we only need characterize an optical medium by its refractive index. V(r) however is not linear in the general case. Nonetheless, the expression for the product V(r)E, also known as the polarization P(t), can be expanded using a Taylor series;

P(t)=κ⁽¹⁾E+κ⁽²⁾E²+κ⁽³⁾*E³+. . . (4)

where κ(ⁿ) is the n^{th} order complex optical susceptibility of the medium. The presence of such a term is generally referred to as an n^{th} order nonlinearity. In general κ⁽ⁿ⁾ is an n+1 order tensor representing both the polarization dependent nature of the parametric interaction as well as the symmetries (or lack thereof) of the nonlinear material. The first term on the right-hand-side (RHS) in Eqn. 4 represents linear interactions between light and matter such as one-photon absorption, reflection and refraction of light. The second term represents sum and difference frequency generation, a special case of which is optical second harmonic generation (SHG). In this case, two photons are removed from the incident light and combined to form a single photon at twice the original frequency. The third term represents a number of third-order nonlinear optical processes, including third harmonic generation.

Consider now a monochromatic laser light with electric field E(t) and a fundamental frequency ωₒ applied to a non-linear material with third-order non-linear susceptibility κ³(t):

E(t)=E cos (ωₒt) (5)

Assuming the material possess only a non-zero κ³(t), the polarization P(t) within the non-linear material consists of only the third-order non-linear polarization, P³(t):

P³(t)=κ³E³(t) (6)

Using the identity:

cos³(ωt)=0.25cos (3ωₒt)+0.75cos (ωt) (7)

yields:

P³(t)=0.25κ³E³(t) cos (3ωt)+0.75κ³E³(t) cos (ωt) (8)

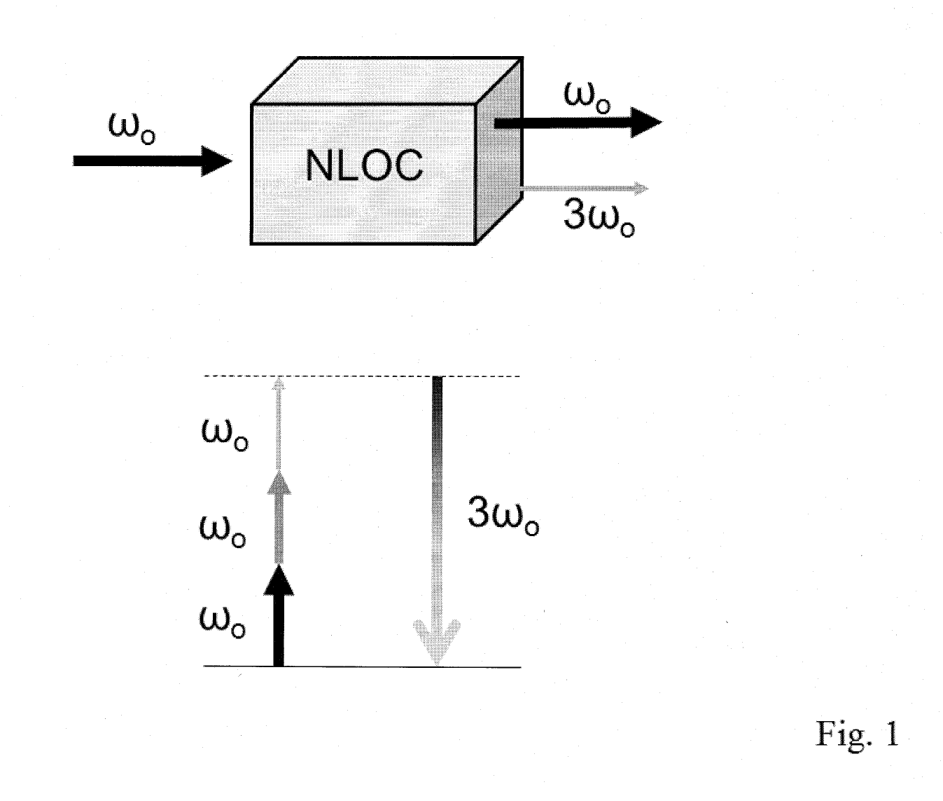

The first term on the RHS of Eqn 8 leads to generation of electromagnetic radiation at an angular frequency of 3ωₒ by a process known as third harmonic generation. The relationship between the fundamental frequency of the laser, the non-linear crystal and third harmonic generation is schematically illustrated in FIG. 1.

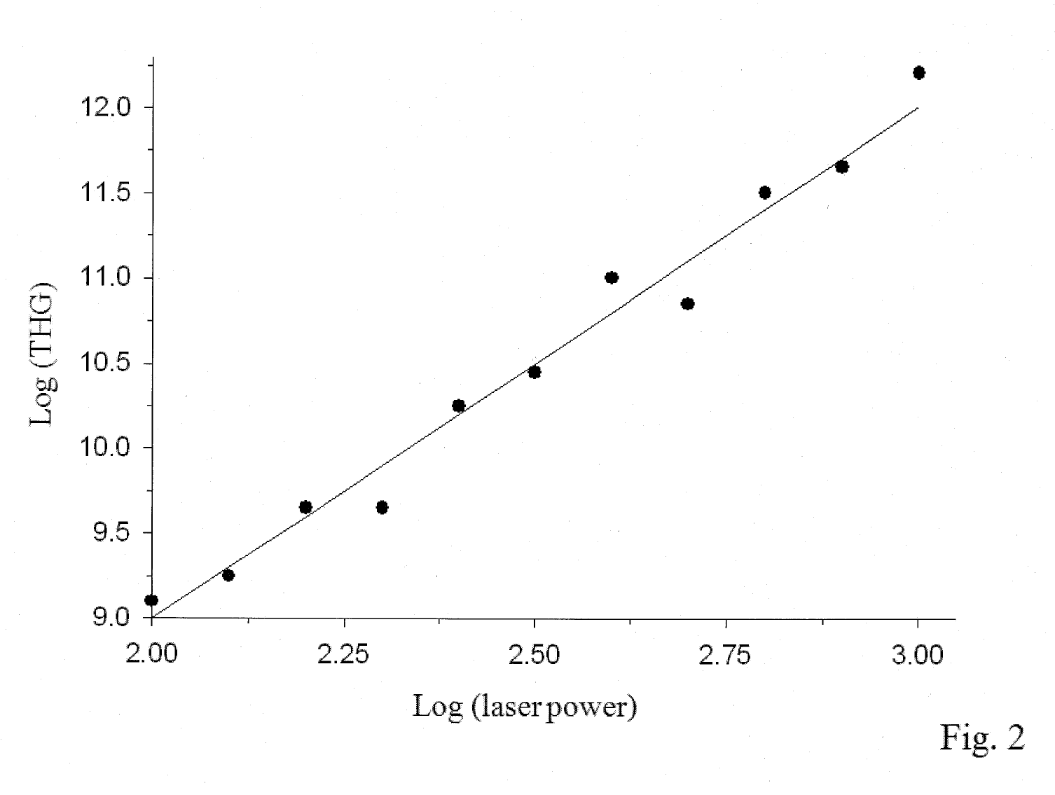

The non-linear optical properties of haemozoin were studied by measuring the intensity of light emitted by haemozoin in solution as function of the intensity of the incident laser light. A log-log plot of the data were fitted with a straight line with a slope of 2.81±0.3 (FIG. 2). This value is very close to the theoretical value of 3, expected from third-power dependence and hence consistent with a third harmonic generation process.

Methods for laser-induced inactivation of malaria parasites are described herein.

All methods make use of pulsed NIR lasers to induce generation of UV light as described in FIG. 1, by haemozoin crystals within the malaria parasites. Since humans do not produce haemozoin the co-lateral damage to the host's cells of irradiation with NIR laser should in principle be low and the therapeutic ratio high.

In one embodiment the patient is being irradiated directly through the skin in one spot (i.e. the arm) with a pulsed NIR laser at a wavelength of 800 nm. Light at 800 nm is relatively harmless to the patient thanks to a dip in oxyhaemoglobin absorption spectrum (16).

In another embodiment the patient's blood is passed through a dialysis machine equipped with a NIR-transpa rent window through which the blood can be irradiated.

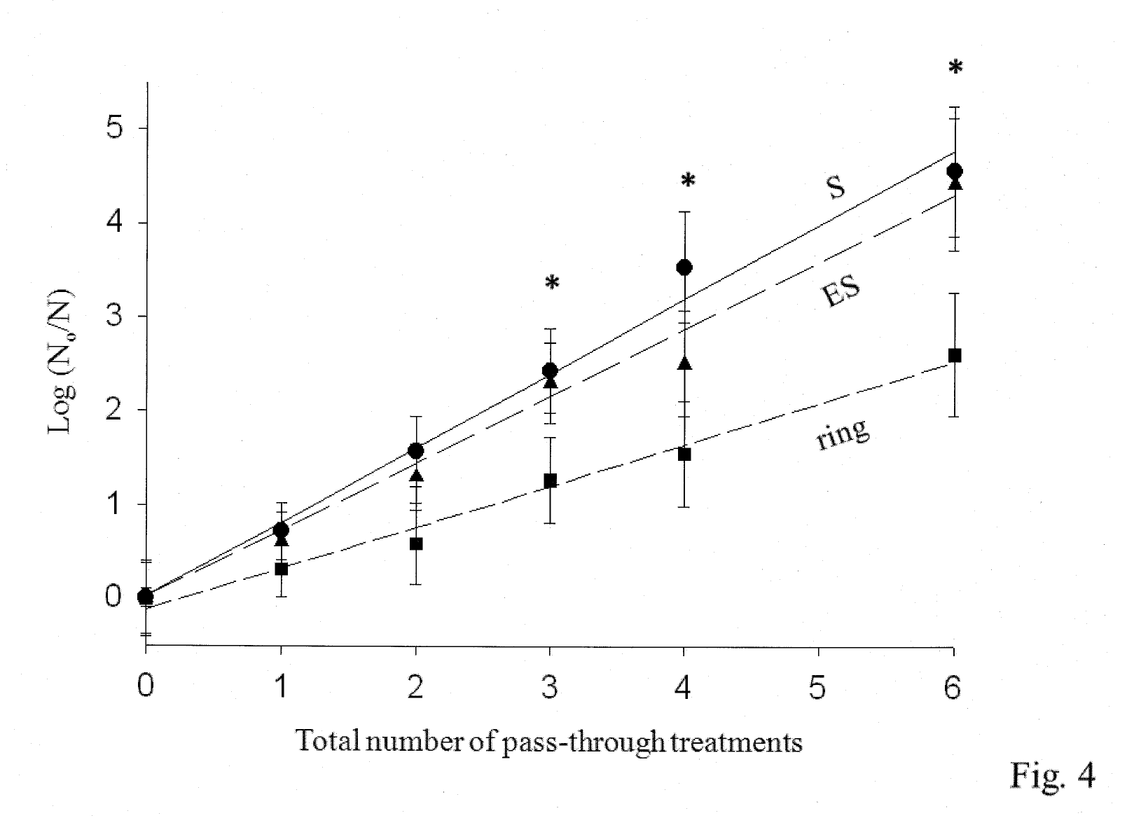

The data shown in FIG. 4 suggest that a ˜0.5-log reduction in parasite count can be achieved by passing the entire volume of the blood sample once through the path of a single laser beam with an intensity of ˜0.5 W/cm². At a standard perfusion rate of 300 ml/min it would take ˜20 minutes to circulate the entire volume of blood of an adult male patient (˜6 liters) through the dialysis machine to achieved a ˜0.5-log reduction in parasitaemia. According to World Health Organization guidelines (17), severe malaria is defined as a case with >250,000 parasites/microL of blood; while mild malaria is defined as a case with <100,00 parasites/microL of blood. Thus, a ˜0.5-log reduction in parasitaemia may be sufficient to downgrade the symptoms of malaria in a patient undergoing treatment, from severe to mild. Based on the linear log kill curves obtained in our studies (FIG. 4) the kill rate can be increased linearly by adding more lasers along the dialysis perfusion line. Thus, a ˜1-log reduction can be achieved in principle by passing the blood through two laser beams in tandem.

### EXAMPLES

### Example 1

### Laser-Induced Reduction in Parasitaemia

In this example I demonstrate that irradiation of infected human erythrocytes, containing the malaria parasite, with pulsed NIR laser inactivates the parasites (15). Plasmodium falciparum HB-3 (ATTC 50113) from a frozen vial was placed in culture and maintained by the continuous flow technique (18). For experiments, cultures were initiated with a 10% suspension of a human A+ erythrocytes in RPMI 1640 medium containing 10% human A+ serum at a starting parasitaemia of 0.2% as described by Waki et al (19). Cultures were incubated in a cell culture incubator at 37 degrees Celsius with a gas mixture containing 5% CO₂, 10% O₂ and 85% N₂. Triplicate cultures (0.5 ml) were prepared in 24-well flat-bottom tissue-culture plates and multiplication of parasites monitored daily using Giemsa-stained thin films made from each of the cultures. For determination of growth ˜10,000 erythrocytes were examined at 1000× magnification under oil.

Two methods were used to synchronize parasites as previously described (19). First, cells were treated three times with D-sorbitol at 0, 48 and 88 hours. The 88 hr treatment selects for a relatively narrow age distribution of newly formed rings (“fine tuning”) (20).

Prior to transition from schizont to ring form the parasites were treated again with sorbitol to obtain young ring form. In the second method, parasites in the stage of DNA synthesis were removed from the culture by treating the cells with 50 mM hydroxyurea for six hours. Parasites in trophozoite stage were prepared by cultivating the young ring form parasite for 18 hours or 30 hours. The 18-hr trophozoites which had just transformed from ring forms and those that remained as trophozoites after 30 hours in culture were hydroxyurea-sensitive and were designated early (ES) and synthesis (S) phases, respectively(19).

For irradiation, 60 ml infected blood cells were loaded onto a sterile reservoir and passed multiple times through a quartz flowcell cuvette with a 6.5 mm wide×6.5 mm high aperture and a 5 mm path length (Starna, Atascadero, Calif.) at a flow rate of 1.0 ml/sec using a peristaltic pump with sterile tubing. Cells were irradiated with a 300 kHz RegA 9050 laser (Coherent, Inc.) pumped by a 10 W Verdi (Coherent, Inc.). The beam had a pulse with of ˜50 fs and the rms output power attenuated to ˜485±15 mW with a neutral density filter. Following irradiation a 1-ml aliquot was diluted with a 10% fresh erythrocyte suspension to provide un-irradiated host cells for the parasite and was put into culture dish and return to standard culture conditions. The number of pRBC was monitored daily and the results plotted as the ratio of the initial number.

The time it took to reduce pRBC count to 37% of its initial value is referred to as the time constant (τ) for parasite inactivation. Tao (τ) was calculated from the slope of the kill curves (the plots of Log(Nₒ/N) vs. time) by assuming a first-order kill reaction kinetics (N=Nₒe^{-τ/τ}). The corresponding energy (Eₒ) of NIR light needed to reduce parasite count to 37% of its initial value was calculated using the relation Eₒ=Pτ, were P is the laser output power at 800 nm. Experimental values for both Eₒ and τ are listed in Table 1.

Growth curves for parasites synchronized as late-stage trophozoites were constructed by inoculating the parasite into cultures. Multiplication of parasites in culture was plotted on log-linear plots and straight-line growth curves were extrapolated on the vertical axis to determine initial parasite counts.

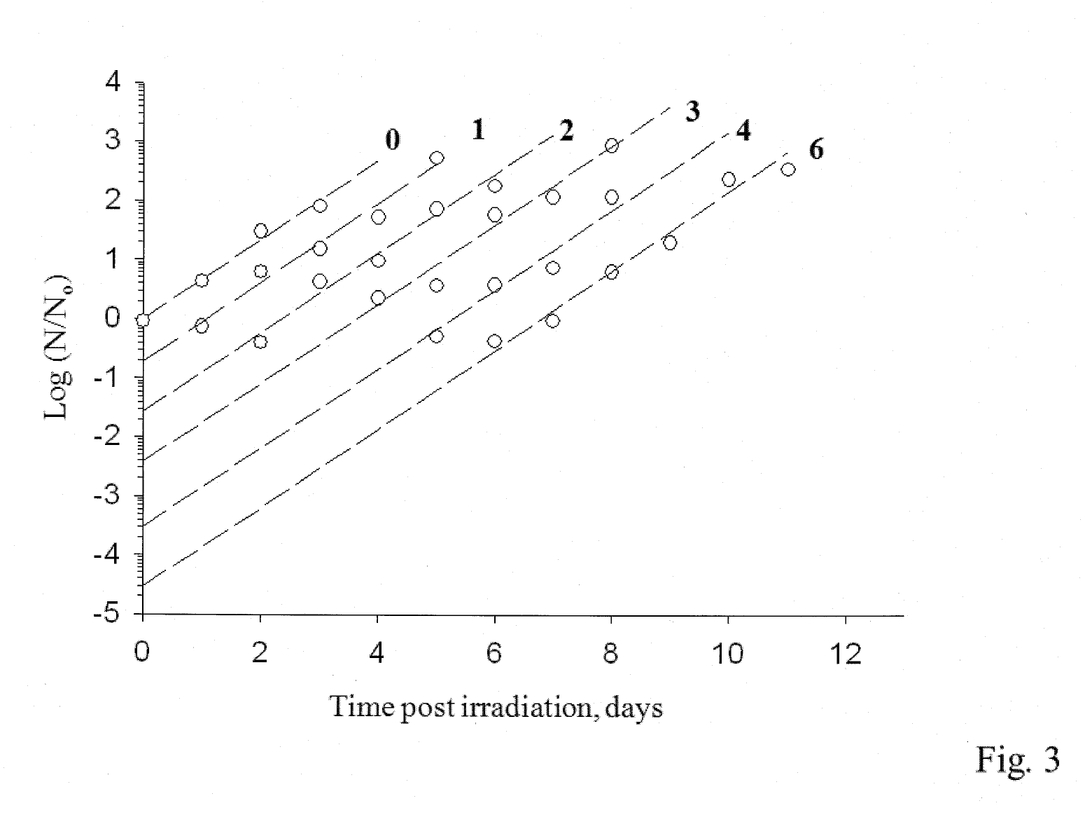

The effect of irradiation on parasite viability was evaluated by plotting growth curves following various irradiation times of machine-circulated blood. Each minute the entire test volume of blood passed through the laser beam once. To determine the survival rate of parasites after irradiation with the laser, the corresponding growth curves of irradiated parasites were extrapolated on the vertical axis as shown in FIG. 3. Reduction in parasitaemia was quantified by fitting the ratio of remaining viable parasites to the initial un-irradiated parasite count with a single exponential decay function. This function produces a straight line on a logarithmic scale. FIG. 4 plots the negative of that log ratio. As can be seen, a ˜4-log reduction in parasite count for the late and early trophozoites phases were obtained following six full passes of the entire blood sample volume through the laser beam. A ˜2-log reduction for the ring phase was obtained for the same irradiation regimen. The dosimetry data are summarized in Table 1.

### Example 2

### Bactericidal Effect of NIR Laser and Haemozoin

In this example I demonstrate that irradiation of synthetic haemozoin in the vicinity of live bacterial cells, kills the bacteria(15). The results of the experiment are consistent with my hypothesis of a laser-induced pathogenic effect of haemozoin via a third harmonic generation mechanism. Escherichia coli (E. coli, ATCC 11775) from an agar slant were inoculated into 6 ml nutrient broth (Becton Dickinson/Difco) and incubated at 37 degrees Celsius in a cell culture incubator. After 18 h incubation, cells (˜1˜10⁸ CFU/ml) were diluted 10⁶-fold into BHI (Becton Dickinson/Difco) broth and placed in a stirred quartz cuvette containing haemozoin or hemin for irradiation. Cuvettes containing 3 ml cell suspension were placed in a cell holder and irradiated with the laser for various time periods at room temperature. Following irradiated cell samples (0.1 ml) were spread onto agar plates containing 1.5 g/l bile salts (Becton Dickinson/Difco) as a selective agent. After 24 h incubation at 37 degrees Celsius, colony counts were performed to determine cell viability.

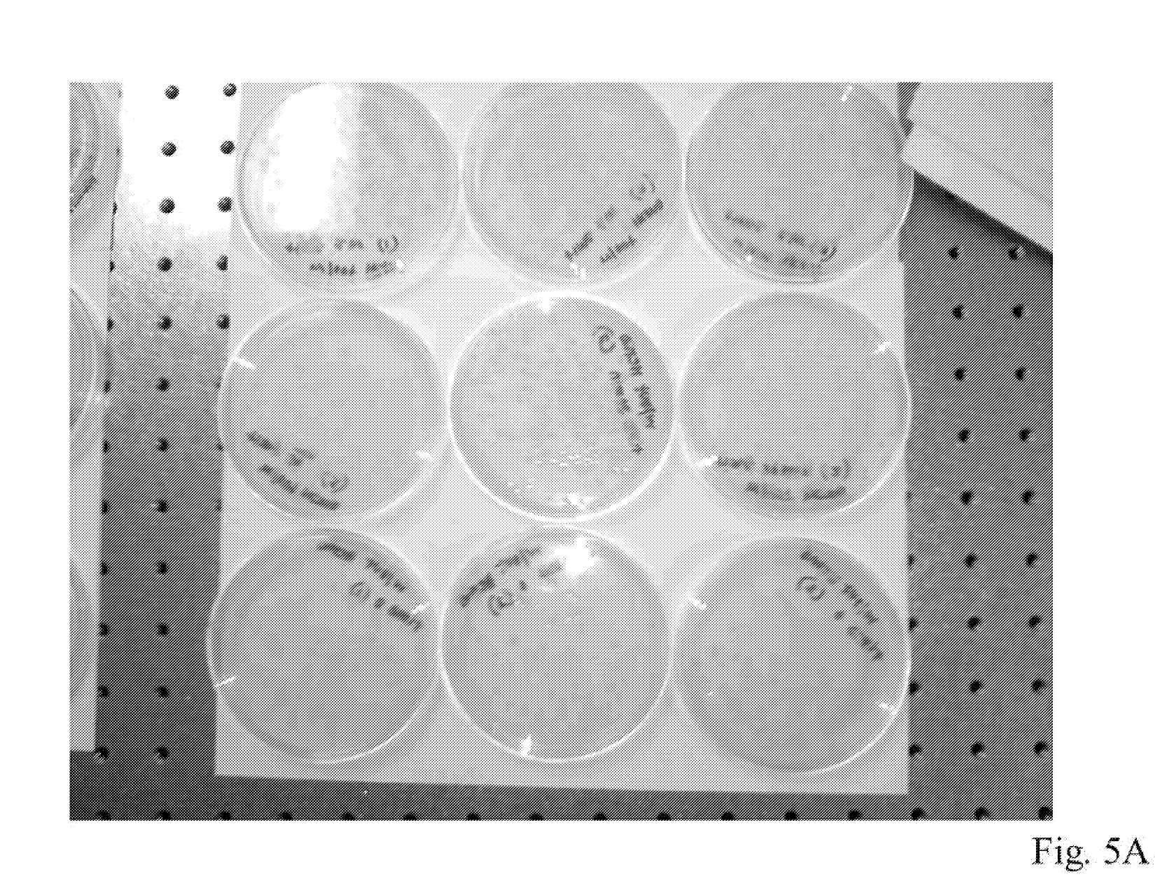

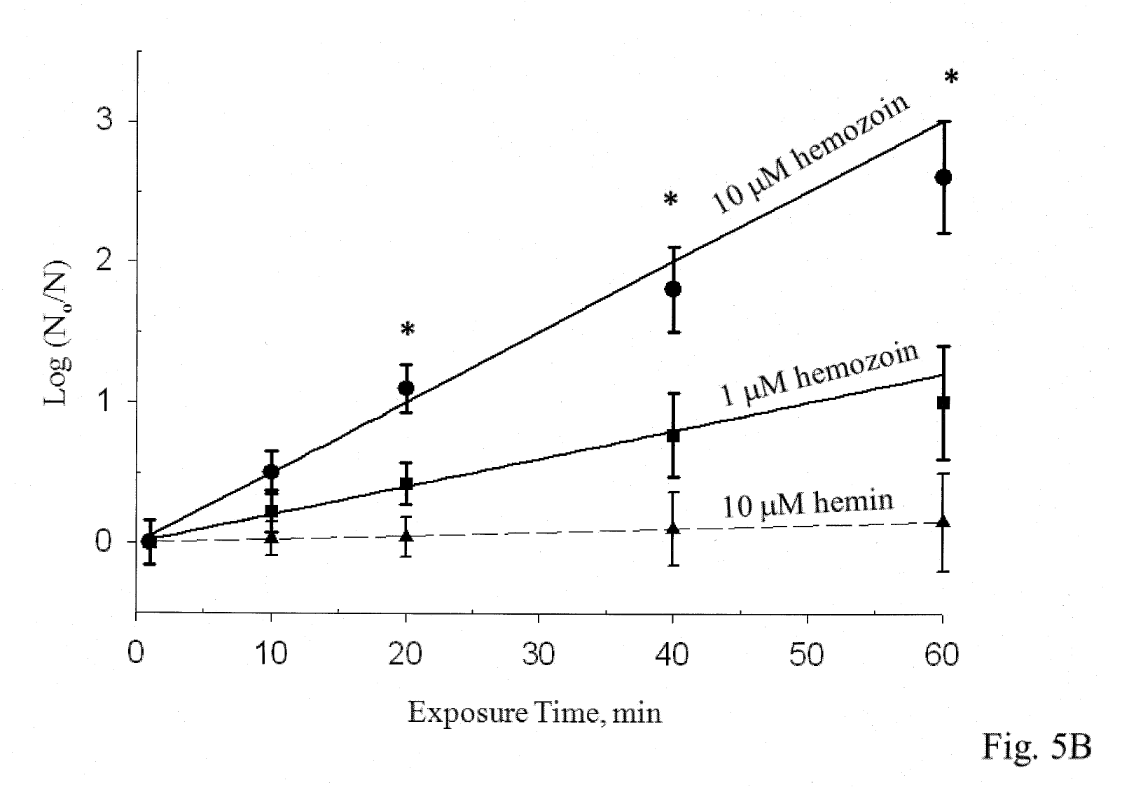

It is hypothesized that parasite kill in our system was caused by haemozoin -mediated UV radiation, causing replication-defective mutations in the parasite's DNA. To gain further insight, synthetic haemozoin crystals were added to a suspension of E. coli bacteria in a cuvette and irradiated the mixture with the laser under continuous stirring. FIG. 5A illustrates the bactericidal effect of the pulsed NIR laser in the presence of haemozoin (1 microM) as a function of exposure time. The full data set for the bactericidal effect of haemozoin are summarized in Figure SB. As can be seen, a ˜1-log reduction in CFU count was obtained with 1 microM haemozoin following 60 min exposure to the laser; and a ˜2-log reduction with 10 microM haemozoin, for the same exposure time.

To further test the hypothesis that haemozoin's bactericidal effect was mediated by UV radiation, due to third harmonic generation, control experiments were carried out by replacing haemozoin with hemin (a precursor of haemozoin). Hemin cannot generate UV light by THG. Illuminating the cells in the presence of hemin induced a moderate cell kill with ≦15% reduction in CFU (FIG. 5B, dashed line), possibly via a photodynamic effect(21). The lack of a significant bactericidal effect upon treatment with hemin suggests that for the most part haemozoin in these experiments remained intact and did not revert to its precursor hemin form when put in solution.

By multiplying the concentration of haemozoin (in femtograms per parasitized RBC) obtained from cultured parasites, by the geometric mean number of parasites per microlitre in patients with mild and severe malaria, Keller et al estimated the blood concentration of haemozoin as ranging from 1.9 microgram/ml in patients with mild symptoms to 12.9 microgram/ml in patients with more severe cases of malaria(22). These concentrations correspond to molar concentrations of 2.9 and 19.7 microM, respectively and are on the same order of magnitude as the concentrations used in our pilot bactericidal experiments.

It should be understood that all embodiments which have been described may be combined in all possible combinations with each other, except to the extent that such combinations have been explicitly excluded.

Finally, nothing in this Specification shall be construed as an admission of any sort. Even if a technique, method, apparatus, or other concept is specifically labeled as “prior art” or as “conventional,” Applicants make no admission that such technique, method, apparatus, or other concept is actually prior art under 35 U.S.C. §102, such determination being a legal determination that depends upon many factors, not all of which are known to Applicants at this time.

TABLE 1
Dose-response data for NIR-laser germicidal effect.

| Organism | τ, sec* | Eₒ, J/cm^{2#} |
|---|---|---|
| Plasmodium falciparum | | |
| Late trophozoites (S) | 34.8 ± 3.9 | 16.6 ± 1.7 |
| Early trophozoites (ES) | 35.6 ± 3.6 | 17.2 ± 1.7 |
| Ring | 60.2 ± 6.4 | 29.1 ± 3.0 |
| Escherichia coli | | |
| +10 microM haemozoin | 711 ± 8.1 | 345.2 ± 40 |
| +1.0 microM haemozoin | (165 ± 14) · 10¹ | 800 ± 8.4 |
| +10 microM hemin | (104 ± 11) · 10³ | (504 ± 51) · 10² |

*τ is the time to reduce parasite count to 37% of its initial value following irradiation with the laser.
^{#}Eₒis the energy of NIR light needed to reduce parasite count to 37% of its initial value.

### References (incorporated herein by reference)

1. Mayor, S. 2008. WHO report shows progress in efforts to reduce malaria incidence. BMJ 337:a1678.
2. Kappe, S. H., A. M. Vaughan, J. A. Boddey, and A. F. Cowman. 2010. That was then but this is now: malaria research in the time of an eradication agenda. Science 328:862-866.
3. Augustine, A. D., B. F. Hall, W. W. Leitner, A. X. Mo, T. M. Wali, and A. S. Fauci. 2009. NIAID workshop on immunity to malaria: addressing immunological challenges. Nat Immunol 10:673-678.
4. Sullivan, D. J. 2002. Theories on malarial pigment formation and quinoline action. Int J Parasitol 32:1645-1653.
5. Le Bras, J., and R. Durand. 2003. The mechanisms of resistance to antimalarial drugs in Plasmodium falciparum. Fundam Clin Pharmacol 17:147-153.
6. Bienzle, U., O. Ayeni, A. O. Lucas, and L. Luzzatto. 1972. Glucose-6-phosphate dehydrogenase and malaria. Greater resistance of females heterozygous for enzyme deficiency and of males with non-deficient variant. Lancet 1:107-110.
7. Ruwende, C., S. C. Khoo, R. W. Snow, S. N. Yates, D. Kwiatkowski, S. Gupta, P. Warn, C. E. Allsopp, S. C. Gilbert, N. Peschu, and et al. 1995. Natural selection of hemi- and heterozygotes for G6PD deficiency in Africa by resistance to severe malaria. Nature 376:246-249.
8. Beutler, E., and S. Duparc. 2007. Glucose-6-phosphate dehydrogenase deficiency and antimalarial drug development. Am J Trop Med Hyg 77:779-789.

9. Usanga, E. A., and L. Luzzatto. 1985. Adaptation of Plasmodium falciparum to glucose 6-phosphate dehydrogenase-deficient host red cells by production of parasite-encoded enzyme. Nature 313:793-795.
10. Mohr, H., U. Gravemann, and T. H. Muller. 2009. Inactivation of pathogens in single units of therapeutic fresh plasma by irradiation with ultraviolet light. Transfusion 49:2144-2151.
11. Cates, E. L., M. Cho, and J. H. Kim. 2011. Converting visible light into UVC: microbial inactivation by Pr(3+)-activated upconversion materials. Environ Sci Technol 45:3680-3686.
12. Valero, A., M. Begum, S. L. Leong, A. D. Hocking, A. J. Ramos, V. Sanchis, and S. Marin. 2007. Effect of germicidal UVC light on fungi isolated from grapes and raisins. Lett Appl Microbiol 45:238-243.
13. Ammermann, D. 1988. DNA damage and repair in Stylonychia lemnae (Ciliata, Protozoa). J Protozool 35:264-267.
4. Belisle. J. M., S. Costantino, M. L Leimanis, M. J. Bellemare, D. S. Bohie, E. Georges, and P. W. Wiseman. 2008. Sensitive detection of malaria infection by third harmonic generation imaging. Biophys J 94:L26-28.
15. Leblanc, D., R. Story, and E. Gross. 2012. Laser-induced inactivation of Plasmodium falciparum. Malar J 11:267.
16. van Gernert, M. C., and A. J. Welch. 1989. Clinical use of laser-tissue interactions. IEEE Eng Med Biol Mag 8:10-13.
17. World Health Organization. 2010. Guidelines for the treatment of malaria. World Health Organization, Geneva.
18. Trager, W. 1979. Plasmodium falciparum in culture: improved continuous flow method. J Protozool 26:125-129.
19. Waki, S., I. Yonome, and M. Suzuki. 1983. Plasmodium falciparum: attenuation by irradiation. Exp Parasitol 56:339-345.
20. Lambros, C., and J. P. Vanderberg. 1979. Synchronization of Plasmodium falciparum erythrocytic stages in culture. J Parasitol 65:418-420.
21. Gross, E., B. Ehrenberg, and F. M. Johnson. 1993. Singlet oxygen generation by porphyrins and the kinetics of 9,10-dimethylanthracene photosensitization in liposomes. Photochem Photobiol 57:808-813.
22. Keller, C. C., P. G. Kremsner, J. B. Hittner, M. A. Misukonis, J. B. Weinberg, and D. J. Perkins. 2004. Elevated nitric oxide production in children with malarial anemia: hemozoin-induced nitric oxide svnthase type 2 transcripts and nitric oxide in blood mononuclear cells. Infect Immun 72:4868-4873.

I claim:

1. An apparatus comprising a pulsed near infra-red (NIR) laser and a dialysis machine equipped with a NIR transparent window for the treatment of malaria by irradiation of the infected perfused blood through the window.

2. An apparatus comprising a pulsed near infra-red (NIR) laser for the treatment of malaria by irradiation of the infected blood through the patient's own skin.
A method comprising claim 1 to treat a person infected with malaria.
A method comprising claim 2 to treat a person infected with malaria.
